(19) United States
(12) Patent Application Publication
(10) Pub. No.: US 2025/0318900 A1
(43) Pub. Date: Oct. 16, 2025

(54) AUTO CONTOURABLE RADIOPAQUE FIDUCIAL MARKER WITHOUT ARTIFACT

(71) Applicant: Videra Surgical Inc., San Francisco, CA (US)

(72) Inventors: Alex Vayser, Mission Viejo, CA (US); Henry Jay Lee, Chappaqua, NY (US); Dominic Parsa Rafie, Austin, TX (US)

(21) Appl. No.: 19/248,137

(22) Filed: Jun. 24, 2025

Related U.S. Application Data
(63) Continuation of application No. 18/048,554, filed on Oct. 21, 2022.
(60) Provisional application No. 63/270,891, filed on Oct. 22, 2021.
(60) Provisional application No. 63/263,033, filed on Oct. 26, 2021.

(51) Int. Cl. A61B 90/00

(52) U.S. Cl. CPC A61B 90/39; A61B 2090/3908; A61B 2090/3966; A61B 2090/3987

(57) ABSTRACT

A radiopaque fiducial marker (RFM) includes an implantable marker element having a density ranging from about 0.8 g/cm³ to about 1.5 g/cm³ such that the implantable marker element is radiopaque but does not create artifacts under magnetic resonance imaging (MRI) or ultrasound imaging. The RFM may be a filament with a radiodensity of between +100 to +2800 Hounsfield Units, or the filament may have a radiodensity of at least 100 Hounsfield Units above the radiodensity of adjacent soft tissue, or a radiodensity of at least 100 Hounsfield Units below the radiodensity of adjacent bony anatomy.

### CLAIM OF PRIORITY

The present application is a continuation of U.S. patent application Ser. No. 18/048,554 filed on Oct. 21, 2022 (Attorney Docket No. 5210.003US1), which application is a non-provisional of and claims the benefit of priority to U.S. Provisional Patent Application Nos. 63/270,891 filed on Oct. 22, 2021 (Attorney Docket No. 5210.003PRV) and 63/263,033 filed on Oct. 26, 2021 (Attorney Docket No. 5210.004PRV); the entire contents of each are incorporated fully herein by reference.

### CROSS-REFERENCE TO RELATED PATENT DOCUMENTS

This patent application is also related to U.S. patent application Ser. No. 16/160,229 (now U.S. Pat. No. 11,413,112) and U.S. patent application Ser. No. 16/791,410 (now U.S. Pat. No. 11,464,998); each of which is hereby incorporated by reference herein in its entirety.

### BACKGROUND

Radiopaque fiducial markers (RFM) are commonly used to mark areas of tissue for radio therapeutic treatment or for radiological observation of area of interest. For example a tissue cavity may be marked after a tumor has been excised so that the area may be monitored and/or treated postoperatively. In another example, a RFM may be used to mark a biopsy site so the patient can be observed and monitored by the radiologist to easily locate the biopsy site at a later time. In other non-limiting examples, it may also be desirable to mark an anastomosis or other regions where tissue diagnosis or treatment has been performed or will need to be performed. So for example, during a colon resection, the area of anastomosis can be marked, so that if there is a leak, the area can be easily imaged by x-ray. In another example, some patients may undergo postoperative radiation treatment where the radiation oncology team may establish a software based treatment plan based on computerized tomography images. The treatment plan can provide the target area of interest for radiation to be delivered to, and minimize toxicity to adjacent tissue. In other radiologic monitoring and treatment situations, ultrasound or magnetic resonance imaging is used. Therefore, the RFM ideally should be compatible with imaging modalities.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an ultrasound image of breast tissue with undesirable artifacts generated by metal RFM's used in the patient.

FIG. 2 shows a magnetic resonance image (MRI) of breast tissue with undesirable artifacts generated by metal RFM's used in the patient.

FIG. 3 shows various organs of the body auto-contoured.

FIG. 4 shows a radiation treatment plan in breast tissue.

FIG. 5 illustrates a seroma in breast tissue.

FIG. 6 illustrates the use of metal clips.

FIG. 7 shows a radiation dose plan for target tissue with clips.

FIG. 8 shows a boost plan.

FIGS. 9A-9C show artifacts from a metal RFM on a CT image.

FIG. 10 shows a continuous RFM disposed around a tumor bed.

FIG. 11 shows a reconstructed 3D image of the continuous RFM from FIG. 10.

FIG. 12 is a CT image of a continuous RFM.

FIGS. 13A-13D illustrate a CT simulation dataset with auto-detection and auto-contouring of the continuous RFM.

FIGS. 14A-14D show an interpolation of FIGS. 13A-13D.

FIGS. 15A-15D show reconstruction of FIGS. 13A-13D and 14A-14D into a volumetric structure.

FIG. 16 shows an example of a monofilament.

FIG. 17 shows an example of a multifilament.

FIG. 18 shows an example of a filament with a suture needle on one end of the filament.

FIG. 19 shows an example of a cavity marked with a RFM.

FIG. 20 shows the cavity of FIG. 19 with tissue re-approximation and movement of the RFM.

### DETAILED DESCRIPTION

### Imaging Artifacts

RFM are commonly used to mark areas of tissue for radio-therapeutic treatment or for radiological observation of an area of interest. This commonly occurs during the surgical excision of diseased or otherwise suspicious tissue. These types of RFM can migrate after implantation, since they are attached to the tissue and can easily get dislodged and therefore must be secured to the target tissue to prevent unwanted movement. It is possible for the RFM to be painted or sprayed directly on to the tissue, however, the radiopacity of the material must remain consistent throughout the length in order for it to be seen at different sections of the tissue. In some cases, the RFM is tied to the target tissue and the marker may have inadequate tensile strength to withstand knotting and therefore the marker may break. Additionally, some RFMs are challenging to observe under radiographic imaging while others, especially metallic, can create unwanted image artifacts under other imaging modalities such as magnetic resonance imaging (MRI) or ultrasound (US). Furthermore, in some examples, the RFM may be discrete markers that are placed individually around the cavity and it can be difficult to define the volumetric aspects of the cavity later when imaged, especially if the cavity is not symmetric since the RFMs have no correlation to each other. Examples of devices disclosed herein address at least some of these challenges. Any of the RFMs disclosed herein may be a RFM filament or a plurality of RFM filaments.

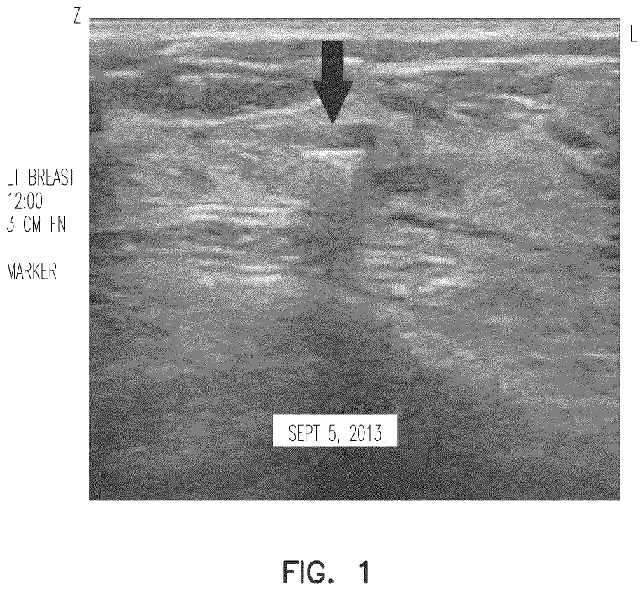

FIG. 1 shows an ultrasound image of breast tissue with undesirable artifacts generated by metal RFMs used in the patient.

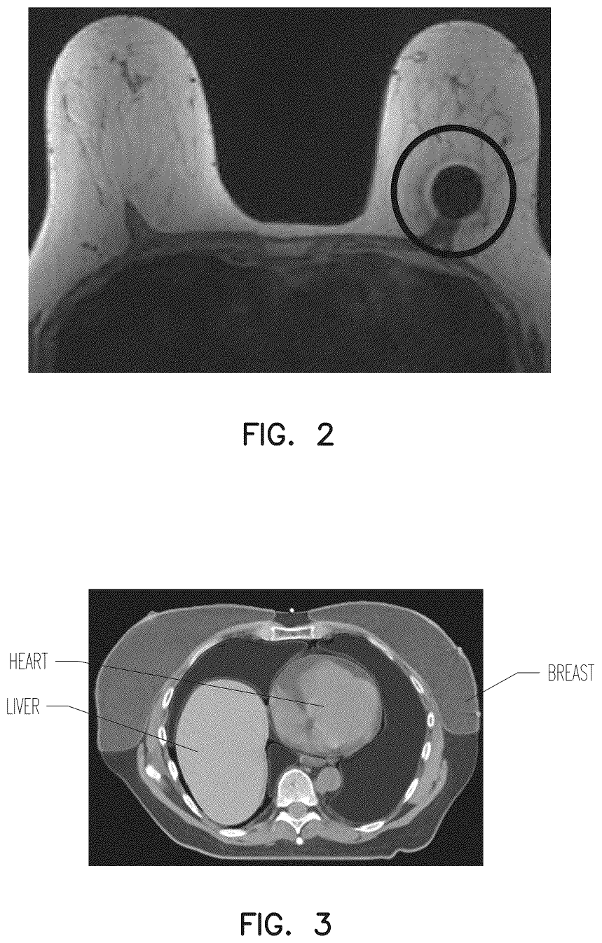

FIG. 2 shows a magnetic resonance image (MRI) of breast tissue with undesirable artifacts generated by metal RFMs used in the patient.

The artifacts can interfere with interpretation, diagnosis and treatment of the target tissue and therefore it is desirable to use a RFM that does not result in imaging artifacts or minimize artifacts.

Examples of various RFMs and their properties are disclosed below and additional information on these markers is disclosed in U.S. patent application Ser. No. 16/160,229 (now U.S. Pat. No. 11,413,112) and U.S. patent application Ser. No. 16/791,410 (now U.S. Pat. No. 11,464,998). The entire contents of each of these patents is incorporated herein by reference. Any of the features, material characteristics or methods described in these patents may be applied to any of the marker concepts described below.

It may be desirable to provide a RFM that is visible under radiographic imaging and that either minimizes artifacts or does not create artifacts under MRI (magnetic resonance imaging) or ultrasound and examples of a marker are described below.

As described in previous patent applications incorporated by reference, marking the site of a surgical resection to identify the location for subsequent radiotherapy treatments is advantageous. Various methods have been used to do so, including doping polymers with radiopaque components to generate a continuous and not a discrete RFM.

Doping polymer materials, such as sutures or catheters with radiopaque compounds allows imaging these devices with x-ray or CT (computerized tomography imaging modalities. For example, as previously described, suture materials can be doped to generate a radiopaque image. There are various suture materials known such as Polypropylene (PP), Polyester, PVDF, Catgut, Polyglactin (Vicryl), Silk, steel or Nylon/polyamide to name a few. Others are known in the art.

Typical doping compounds available are Barium Sulfate (BaSO₄), Tantalum Oxide (Ta₂O₅), Tungsten Oxide (WO₃), Tungsten (metallic), Bismuth Subcarbonate (Bi₂O(CO₃)), Bismuth Oxide (BiO₃) and Bismuth Oxychloride (BiOCl). Others are possible and this list is not intended to be comprehensive or otherwise limiting.

Depending on the mass fraction of the dopant, one can generate various levels of opacity. The tradeoff is higher doping material mass fraction increases radiopacity but will reduce the tensile strength of the filament material thus risking the material to be torn during a procedure such as the knotting process for example, or the sewing process during suturing. Reducing the dopant increases the tensile strength but then reduces radiopacity. So there is a balance to the amount of doping one would utilize to balance opacity and tensile strength. One can also look at other factors of the doping materials to determine optimal choice.

For example, atomic number can be considered to properly identify a material that can yield high opacity with low mass. From Table 1 for example, BaS0₄ has very low atomic number which would require higher mass to be used compared to, for example, Tantalum Oxide.

From the atomic number consideration alone, barium sulfate is the least efficient compound as radiopacity scales with nuclear size. At intermediate to high energies, Compton scattering is proportional to Z, the atomic number. However, at lower energies, the absorption coefficient is dominated by the photoelectric effect and thus proportional to higher powers of the atomic number, varying between 4 and 5. Medical imaging is mainly focused within the energy range [100-100 k] eV, which falls well within the photoelectric effect dominated regime.

From Table 1, the following characteristics: Atom, Atomic Number, Molecular Mass, Atom/Molecule, Molecular Density and Atomic mass are all properties of doping agents. Others are defined below.

TABLE 1

| | | | | | | | | | Density |
|---|---|---|---|---|---|---|---|---|---|
| | | | | | | | | | Corrected |
| | | | Molecular | | Molecular | | | Effective | Effective |
| | | Atomic | mass | Atom/ | Density | Atomic | Opacity | Opacity | Opacity |
| Agent | Atom | Number | (g/mol) | Molecule | (g/cm³) | mass (g) | Ratio | Ratio | Ratio |
| Barium sulfate, | Ba | 56 | 233 | 1 | 4.5 | 137 | 1 | 1 | 1 |
| BaSO₄ | | | | | | | | | |
| Bismuth | Bi | 83 | 260.5 | 1 | 7.36 | 209 | 4.8 | 4.8 | 3.0 |
| oxychloride, BiOCl | | | | | | | | | |
| Bismuth oxide, | Bi | 83 | 466 | 2 | 8.9 | 209 | 4.8 | 9.7 | 4.9 |
| Bi₂O₃ | | | | | | | | | |
| Bismuth | Bi | 83 | 510 | 2 | 6.9 | 209 | 4.8 | 9.7 | 6.3 |
| subcarbonate, | | | | | | | | | |
| Bi₂O₂(CO₃) | | | | | | | | | |
| Tungsten, | W | 74 | N/A | N/A | 19 | 184 | 3.0 | 3.0 | 0.7 |
| (metallic) | | | | | | | | | |
| Tungsten oxide, | W | 74 | 232 | 1 | 7.16 | 184 | 3.0 | 3.0 | 1.9 |
| WO₃ | | | | | | | | | |
| Tantalum oxide, | Ta | 73 | 442 | 2 | 8.2 | 181 | 2.9 | 5.8 | 3.2 |
| Ta₂O₅ | | | | | | | | | |

Opacity Ratio (OR)—is the ratio of two materials using atomic numbers to the 4^{th} power. For example, comparing Bi₂O₃ to BaSO₄:
BaSO₄ atomic number is 56
Bi₂O₃ atomic number is 83
   Opacity Ratio=83⁴/56⁴=4.746×10⁷/9.834×10⁶=4.8 so, Bi₂O₃ is 4.8 times more radiopaque than BaSO₄.

Effective Opacity Ratio (EOR) is OR x atoms/molecule. Therefore, from the previous example, BaSO₄ has 1 Ba atom/molecule of barium sulfate and Bi₂O₃ has 2 Bi atoms per molecule of Bismuth oxide. Thus, 4.8×2/1=9.7. So the effective opacity ratio, EOR of Bi₂O₃ to BaSO₄ is 9.7 and this suggests that Bi₂O₃ is 9.7 times more radiopaque than BaSO₄ and more atoms per molecule yields higher radiopacity. We only consider atoms/molecule that contribute to radiopacity. So in the case of BaSO₄ only Ba contributes to radiopacity, thus it is only 1 atom/molecule. In the case of Bi₂O₃, Bi contributes to radiopacity, therefore, there are 2 atoms per molecule.

Density Corrected Effective Opacity Ratio (DCEOR) is the EOR x molecular density ratio. So based on the previous example, BaSO₄ has molecular density of 4.5 and Bi₂O₃ has molecular density of 8.9, as shown in Table 1. Therefore, the DCEOR is calculated as, 9.7×4.5/8.9=4.9. The denser molecular dopants (e.g. bismuth oxide) are penalized because they alter the density of the base material and dopant the most.

Following the Z⁴ rule describe above, tantalum oxide (Ta₂O₅) would be 2.9 times more radiopaque than barium. However, due to the presence of two tantalum atoms per molecule of dopant, the effective opacity would thus be 5.8 times larger than that of barium sulfate.

Aside from radiopacity, we also want to consider the elimination of artifacts when the RFM is imaged with ultrasound (US) or MRI. These artifacts can obstruct the image and potentially can impact the interpretation by the clinician. Both US and MRI are considered diagnostic modalities. So having a RFM such as a filament which generates artifacts, especially with MRI is not desirable. So there is an advantage of creating a RFM where as a filament that is only visible with x-ray or CT imaging but not visible with US or MRI.

Human tissue ranges in density from 0.9 g/cm³ for fat to 1.07 g/cm³ for muscle, with most organs measuring around 1.05 g/cm³ and bone structure being an outlier at 1.5 g/cm³. For reference, water has a density of 1.0 g/cm³. On the other hand, metals used in the medical industry show densities ranging from 4.5 g/cm³ for titanium, to 8.8 g/cm³ for nickel, with various alloys of steel sitting at 7.9 g/cm³ and nitinol, an alloy of titanium and nickel measuring 6.45 g/cm³. RFM made of those materials are known to show intense echoes under ultrasound imaging and potentially create enough artifact to obscure the diagnostic image.

Therefore creating a doped RFM with densities below 1.5g/cm³ may be desirable as they would be compatible with human tissue.

Many common suture materials mentioned earlier such as Silk, Nylon and polypropylene have densities of 0.85, 1.1 and 0.9 g/cm³ respectively. So these values represent the lower limit (before doping) for the density. Adding dopant to these materials increases the density.

For example using Polypropylene and BaS0₄:

The marker material in this example is produced by doping polypropylene (PP) to 45% w/w with fine barium sulfate (BaSO₄) powder. Because of the large discrepancy in density between the two components, the resulting added volume of BaSO₄ is rather small: the volume ratio of a 45% BaSO₄/PP mixture is 14% as shown below.

Given a total mass of a marker filament of 15 g. For a volume of PP, if 55% of PP is 8.25 g and for the volume calculation we take weight/density, thus 8.25 g/0.9 g/cm³ yields PP volume=9.2 cm³

For a volume of BaSO₄, if 45% of BaSO₄ is 6.75 g, the volume is BaSO₄ 6.75/4.5=1.5 cm³.

The Total Volume (9.2+1.5)=10.7 cm³, and % Vol=1.5/10.7=14%

Thus, the density of the final product would be 15 g/10.7 cm³=1.4 g/cm³.

As shown in the example above, the addition of radiopaque material does increase the overall density but stays within the upper end of the density range for human tissue while being markedly lower than that of metals and alloys. As such, the formation of ultrasound artifacts is minimized in comparison to the signature of metallic markers.

To further refine the analysis, because the aim is inducing radiopacity while maintaining a low overall density, the atomic number ratio can be normalized to the density of the compound (heavier compounds affect overall density more). Therefore, following this analysis, for example, Ta₂O₅ is penalized by its much larger density and it brings down its DCEOR to 3.2 times that of barium sulfate (from 5.8 times, see calculation above and Table 1).

If we wanted to estimate how much Ta₂O₅ is needed in order to have the same radiopacity as the previous example of 45% BaSO₄, this latest result would then predict a PP filament doped with (45/3.2)=14% w/w Ta₂O₅ would be as radiopaque as a PP filament doped with 45% w/w BaSO₄. Hence, the mass percentage is directly related to the radiopacity of the dopant.

Replicating the procedure above to estimate the density of 14% w/w Ta₂O₅/PP, for example. Density is calculated as follows.

Given a total mass of a RFM filament of 15 g, 86% of PP yields 12.9 g and 12.9 g/0.9 g/cm³=14.3 cm³ (Volume of PP).

Furthermore, 15 g-12.9 g=2.1 g Ta₂O₅. So, 2.1 g/8.2 g/cm³=0.26 cm³ (Volume of Ta₂O₅).

So, total Volume (14.3+0.26)=14.56 cm³. The density of the final product would be 15 g/14.56 cm³=1.03 g/cm³.

So, in some examples it may be desirable to pick a dopant that yields product density between 0.8-1.5 g/cm³ and nominally close to 1.0 g/cm³. Any subset of density within this range may be used. For example, 0.8 g/cm³ to 1.39 g/cm³ and greater than 1.4 g/cm³ but less than 1.5 g/cm³ may be used. Or for example, 1.4 g/cm³ plus 3% (1.4 g/cm³ plus 0.04 g/cm³=1.44 g/cm³) may be included or excluded from the range of 0.8 g/cm³ to 1.5 g/cm³. Or, in another example the density may be 0.8 g/cm³ to 1.4 g/cm³. Some of these choices also may be affected by the manufacturability of the dopant with the polymer. So although BaSO₄, may not be as efficient, it has positive manufacturability properties.

As shown in the examples above, the addition of radiopaque material does increase the overall density but stays within the upper end of human tissue range while being markedly lower than that of metals and alloys. As such, the formation of ultrasound artifacts is minimized in comparison to the signature of metallic markers.

Similar to ultrasound, MRI imaging is a diagnostic tool. It may be desirable that implants, specifically biopsy clips or RFM filaments do not create an artifact that can obstruct the view during the diagnostic process. So it also may be desirable to develop tumor bed targeting materials that do not create MRI artifacts.

Barium is a metal of high atomic number (Z=56). In comparison, commonly used metals in the medical industry (mostly 4^{th} period metals like iron, chromium, and titanium) have atomic numbers in the mid-20 s. As such, a smaller overall quantity of barium is required to achieve a comparable radiopacity.

Due to the high reactivity of metallic barium, the salt barium sulfate is used instead. The salt is neither electrically conductive nor ferromagnetic, like polypropylene, and therefore the resulting filament does not generate artifacts under MRI: the filament is not only invisible but it also does not generate signal artifacts. This is contrary to most alloys used in the medical industry which, being ferromagnetic, are known to generate strong artifacts under MRI.

The magnetic susceptibility of BaSO₄ is −65.8, and that of titanium is +151 (in CGS units of 10⁻⁶ cm³ mol⁻¹). By contrast, both nickel and iron (steel) are ferromagnetic. Substances with negative magnetic susceptibility are termed diamagnetic. Barium sulfate, like water and human tissue, is diamagnetic.

Titanium, with its positive magnetic susceptibility is termed paramagnetic. Diamagnetic (or paramagnetic) substances do not show magnetic properties in the absence of a magnetic field, and they are not only safe under MRI, but also tend to not generate strong artifacts.

Some substances' magnetic susceptibility (in units of 10⁻⁶ cm³ mol⁻¹) are listed below. Knowing that titanium's paramagnetic property does not generate significant artifacts under MRI, we can assume that the compounds listed below would be safe under MRI.
Bismuth: −280 (diamagnetic)
Iodine: −88
Barium: −66
Zinc: −16
Water: −13
Copper: −10
Tungsten: +59 (paramagnetic)
Titanium: +151
Tantalum: +154

Therefore, having a polymer with doping material that is diamagnetic or paramagnetic would yield little to no artifacts with MRI imaging, compared to ferromagnetic materials which should completely be avoided in order to prevent artifacts during imaging. A RFM having any of the densities disclosed herein and that is either paramagnetic, diamagnetic, or otherwise non-ferromagnetic may be desirable to provide a RFM that is visible under radiography without creating artifacts under MRI or ultrasound. Or the marker may simply be either paramagnetic, diamagnetic, or otherwise non-ferromagnetic so it does not create MRI or ultrasound artifacts. The absolute value of magnetic susceptibility may be less than 154 in order to avoid artifacts. Any dopants used in a radiopaque fiducial marker may be paramagnetic or diamagnetic with the absolute value of magnetic susceptibility less than 154.

### Automated Computerized Tomography Contouring (Auto Contour)

For patients undergoing postoperative radiation treatment, the radiation oncology team may perform computerized treatment planning based on computerized tomography (CT) images.

This specification will focus on treatment of the breast, but this is not intended to be limiting. The techniques disclosed herein may be applied to any target tissue for radiation or other treatments.

On postoperative CT images, the radiation oncology team creates: 1) contours of normal organ areas to avoid irradiating the normal organs; and 2) a contour of the area(s) that requires targeting to receive radiation therapy. The normal organs are contoured either automatically by a computer through machine learning-based algorithms or manually by members of the radiation oncology team. On the other hand, the target of radiation therapy is usually manually contoured and not automatically contoured. Newer software and systems are now being developed and used for auto-contouring the target treatment areas. Contouring marks the boundaries of the organ or target tissue to be avoided, or to be treated.

For breast cancers, the entire breast is sometimes part of the target of treatment area and thus can be contoured either automatically or manually (note that for breast cancer the target typically includes the site of surgery, the tumor bed, and has historically been manually contoured as discussed below). For automatic contouring, generally whole organs are identified through Al/machine learning. Various techniques are deployed through use of large data sets to identify and separate organs as seen in the FIG. 3 which shows the breasts outlined in purple, liver outlined in yellow, and heart outlined in light purple.

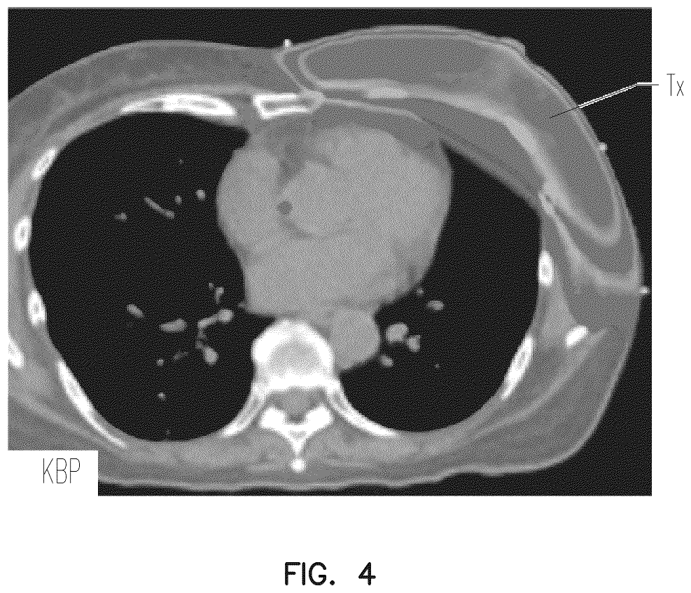

Once the organs and targets are contoured, a computerized treatment plan may be designed based on those contours. A whole breast is treated, Tx, as seen in FIG. 4. The various shading in the treatment zone depict varying radiation intensity.

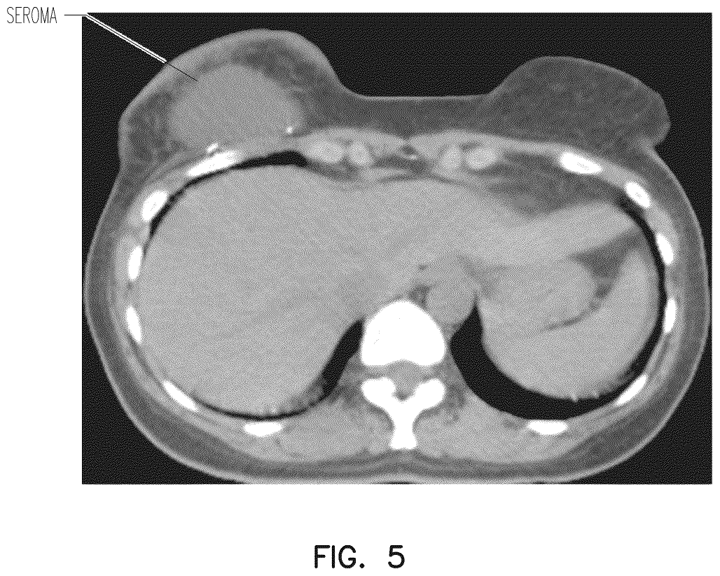

If the tumor bed is a focus of treatment (e.g., boost or partial breast irradiation), the location of the tumor bed needs to be included in the plan. Sometimes this location is visible when a seroma (a fluid-filled area) is left behind. As seen on the breast on the left side of the image in FIG. 5, a seroma is very obvious, so the contour can be drawn around that. Also, the seroma is a crude approximation of the tumor bed, since outlining it overestimates the size and location of the original tumor. However, if there is no seroma present, for example on the breast on the right side of the image FIG. 5, it is extremely complicated to decide where to give the boost.

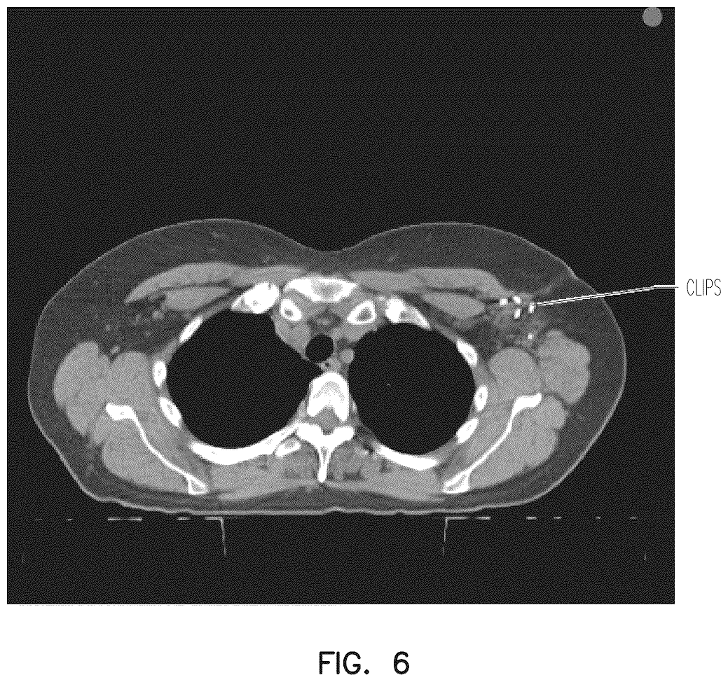

In situations where there is no seroma present, the radiation treatment team has to rely on RFM-like metal clips as seen in FIG. 6 on the right part of the image of the breast. Clips are placed during the surgical excision of the tumor, as illustrated in FIG. 6.

At that point, the medical team has to estimate the radiation target by identifying individual clips and how they define the tumor bed post resection. Using this method the radiation plan will encompass the clips and is thus presumed to encompass the tumor bed. This is a manual process that requires significant time and is not easily automated. Thus it can vary from one radiation oncologist to another as this is all manually interpreted.

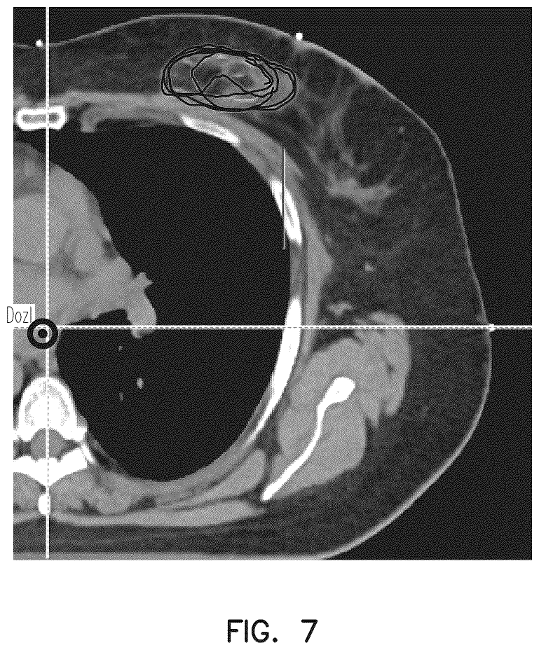

As seen in FIG. 7, the patterns show the plan for various radiation dose levels around the clips. The various shades of circles/ovals depict various range of radiation dosage.

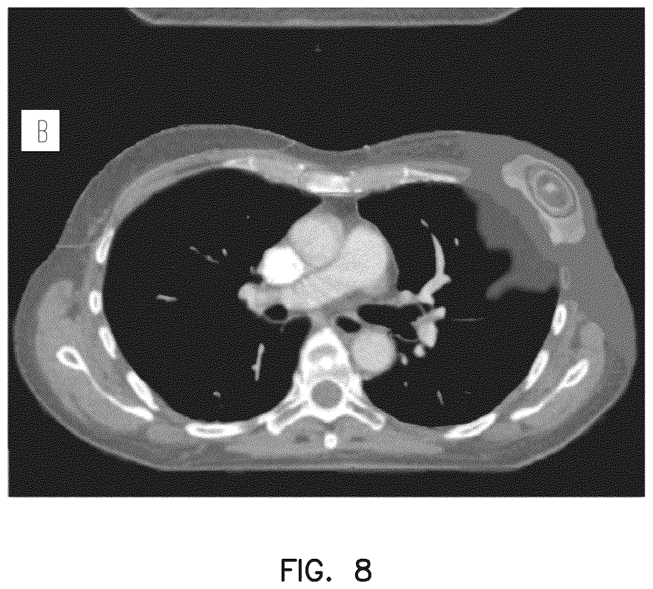

FIG. 8 illustrates an image of an example of a boost plan. The boost directs an extra, concentrated dose or radiation directly to the area where the tumor was surgically removed in order to reduce the likelihood of cancer recurring. So if the treatment plan of the tumor bed does not match closely with the actual resected area, healthy tissue will receive additional, unnecessary toxicity. The boost plan shows the intensity profile. The darker, more focused area is the highest intensity.

There is an opportunity to address the issue of manual estimation by creating an automated process for contouring the tumor bed of resected tumor tissue and not just simply the entire organ (e.g. breast). Individual clips are not sufficient to accomplish this since they do not have a clear correlation between each other in 3D space, thus the treatment plan has to surround the clips as seen earlier. There is also a challenge in auto identifying the clips that surround the tumor bed as they have similar Hounsfield Units (HU) similar to other structures like bone and other biopsy clips or metal wires in the area.

Images of various tissues that appear on a CT scan are quantified by a range of numerical values based on radio density, known as Hounsfield Units (HU). HU are a dimensionless unit universally used in CT scanning to express CT numbers in a standardized and convenient form. HU are obtained from a linear transformation of the measured attenuation coefficients. The physical density of the tissue is proportional to the attenuation/absorption of the beam used during the CT. Denser tissues have greater beam absorption, more positive HU, and brighter (whiter) voxels, whereas less dense tissues have less absorption, lower (possibly negative) HU, and darker voxels.

For any reconstructed CT volume, the HU value at a voxel of interest can be calculated as follows:

$$HU = 1000 ( {μ}_{Measured} - {μ}_{Water} ) / {μ}_{Water} \qquad Equation ( 1 )$$

where, μ_{measured} is the effective linear attenuation coefficient of the voxel under consideration, μ_{Water} is the effective linear attenuation coefficient of water measured under the same imaging conditions. Generally, CT images therefore use 12-bit images, which are able to store values between −1024 and +3071, with metals such as titanium being close to +3000.

Below are some examples of various tissue types and materials and ranges of their HU:
Air: −1000
Water: 0
Iodinated CT contrast: +100 to +600
Iosimenol 340 (type of iodinated contrast): +176 to +264
Iodixanol 320 (type of iodinated contrast): +160 to +256
Small Titanium Clips: +598 to +738
Large Titanium Clips: +2058 to +2437
Lung: −500, Lung Parenchyma: −910 to −850
Fat: −100 to −30
Adipose tissue: −150 to −20
Bone: +700 to +3000
Muscle: +10 to +55
Blood: +30 to +80
Skin: −30 to +60
Kidney: +20 to +50
Liver: +40 to +90
Breast and fatty tissue: −210 to −100 and −99 to +150 for fibro-glandular tissue
Spleen: +35 to +65
Pancreas: +55
Tendon: +75 to +115
Brain: Gray matter +35, White Matter +25

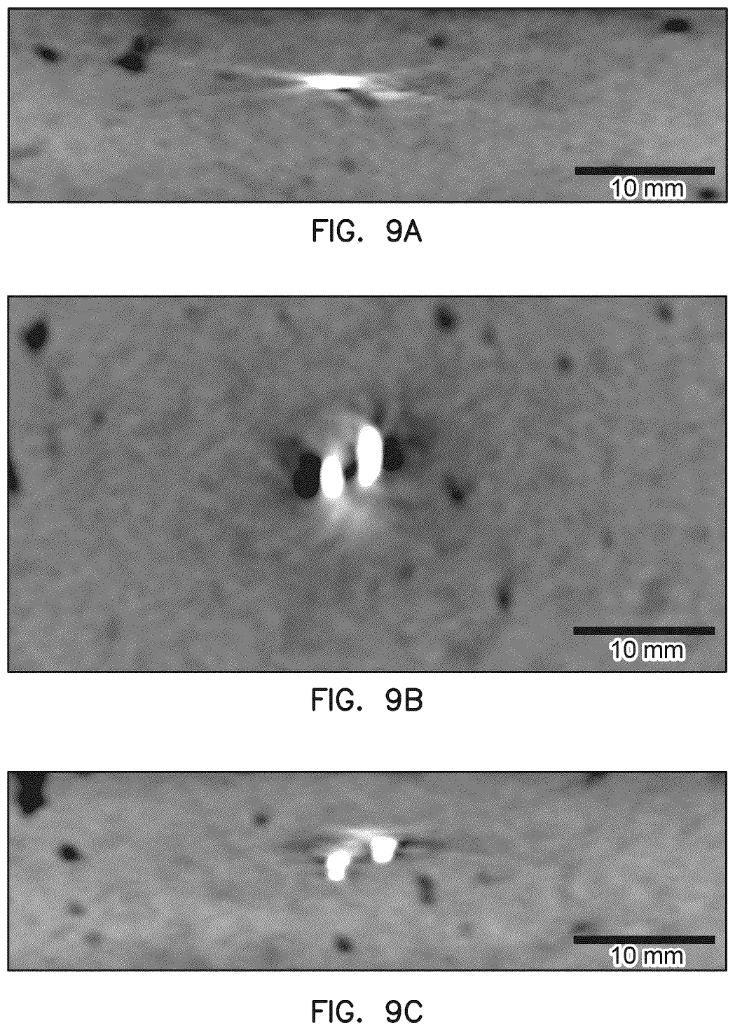

FIGS. 9A-9C illustrate that metal clips may also introduce image artifacts within the CT due to much higher HU than tissue, being around +3,000 HU.

This is where a continuous RFM such as a RFM filament may be designed to delineate the tumor bed or deployed on target tissue for example anastomosis zone based on HU relative differences between the RFM filament and the surrounding breast or other soft tissue without creating artifacts. The marker can also be multiplane and either be threaded to tissue or be attached to the tissue by other means such as clips.

FIG. 10 is an image of a continuous, flexible, multi-plane filament marker that is deployed in the tumor bed to delineate the planes of the tumor bed. Here the filament marker is spirally wound around the tissue walls/perimeter of the cavity. The leading and trailing ends of the filament may be tied or otherwise knotted to the tissue to prevent unwanted movement. Thus, the filament which may be any of the filaments described herein, may pass through a plurality of planes and is continuous. The RFM filament can also be deployed in a single plane.

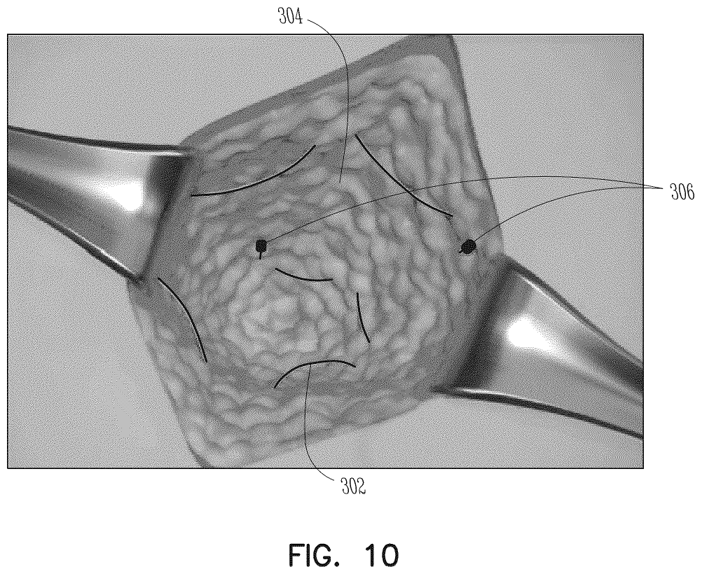

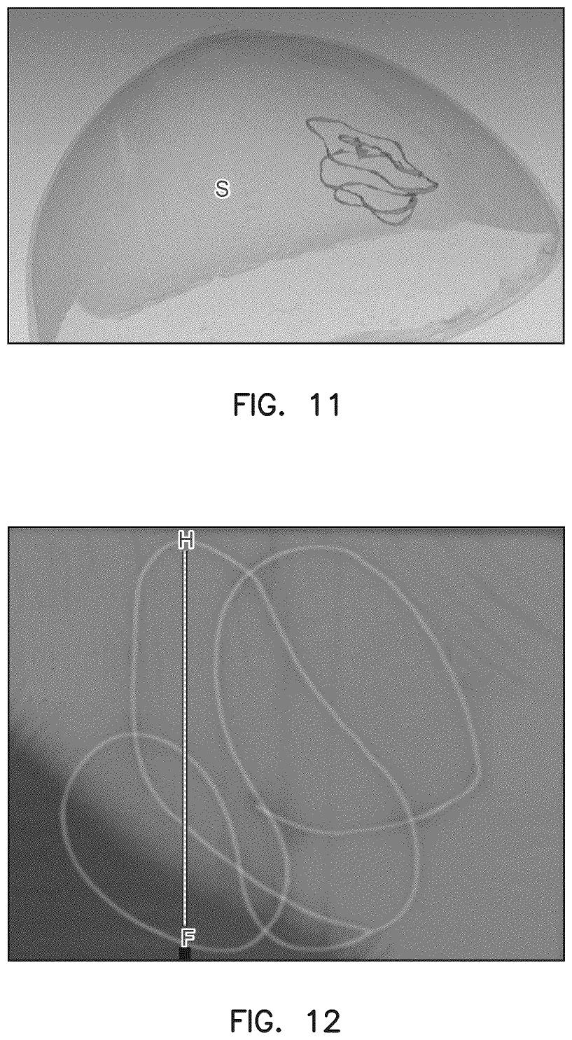

So, when the cavity is imaged with the CT, the continuous RFM looks like FIGS. 11-12 which is a reconstructed 3D image (FIG. 11) and FIG. 12 is the actual RFM filament imaged with CT scanner.

An example in FIG. 10 is a BaSO4 doped Polypropylene filament less than 0.5 mm in diameter and 45% of dopant by weight. The filament has a cross-sectional HU value between +104 to +395 when measured on a phantom with a 0.625 mm CT resolution (controlled environment). However, in practice most patients are scanned with the CT resolution of about 2.5 mm. The decrease in the CT resolution could potentially cause HU averaging within a voxel. The range of cross-sectional HU values of the filament is +100 to +750 and breast tissue for all CT resolutions are −140 to +130 respectively. Since the diameter of the filament is less than 2.5 mm, the HU of the filament within the voxel will be averaged with the surrounding breast tissue in all three dimensions (x, y, and z).

Since there are various ways to develop a RFM filament, whether using BaSO4 or other dopants like Bi₂O₃ or metal wires like nitinol or titanium, in some situations it may be desirable to have HUs greater than soft tissue and not create artifacts. We propose an elongated, flexible continuously RFM filament with HUs between +100 to +2800. Since we know that the HUs will change based on CT resolution, an example at 2.5 mm CT resolution is between +460 to +1985.

Another way to look at this, since the CT resolution and imaging modalities differ from facility to facility, the difference between the average HU of filament minus the average HU of surrounding tissue should be at least 100 HU. Therefore, the filament is preferably at least 100 HU above the surrounding soft tissue. In the case of bony anatomy, the filament is preferably at least 100 HU below than the surrounding bony anatomy.

So the elongated continuous RFM filament needs to have a higher HU compared to soft tissues and lower than the bone. The goal is to design a RFM that can be easily differentiated from soft tissue. It is desirable that it also not be confused for a bony landmark and preferably continuous and have consistent radiopacity and HU during its length.

With a continuous RFM filament, an operator can determine the shape of the tumor bed since its deployed along the walls of the cavity. Although this is helpful to perform a manual contour process to simply trace the marker, there are advantages to automate this process with software and specifically AI (artificial intelligence) and Machine Learning (ML).

As mentioned before, there are technologies that automate the process by contouring organs as seen in the images previously described. However, there is no automated way to contour the tumor bed cavity since the cavities are generally non-symmetric and a significant number of clips would have to be deployed to replicate the shape of the cavity. Continuous RFM filaments may solve this issue, but it is important to understand how the filament appears on the CT image in order for auto contouring software to identify it and differentiate it from the neighboring soft tissue or bone.

For automation, we need to first identify the filament, and the software system will need to scan through all of the Digital Imaging and Communications in Medicine (DICOM) images and mark the filament.

Since most patients' CTs are performed with about 2.5 mm resolution, the filament may not appear continuous in the CT slices. The software needs to interpolate between the images and make the filament continuous. It is desirable for the software to “draw” the entire length of the filament.

The software needs to interpolate between the images and make the filament continuous.

Once the segments are identified and it is interpolated, the volume that the 3D model encompasses can be computed by the treatment planning software. This volume will identify the tumor bed.

Below is an example of a summary of the workflow that may be used for auto contouring.
a) Obtain simulation CT with the imaging protocol that is standard for radiotherapy (RT).
b) Import the CT dataset into an auto-segmentation application.
c) Instruct the application to create a preliminary filament contour via auto-segmentation based on HU range for the filament and then interpolate to create a continuous filament contour.
d) Instruct the application to transform the filament contour into a solid structure. This structure is the tumor bed target for radiation.

FIGS. 13A-13D illustrate a Simulation CT dataset (DICOM)—Auto-segmentation of filament: this leverages the difference in HU of the filament (higher) compared to the surrounding breast tissue (lower HU value). As seen in the upper left (FIG. 13A), lower left (FIG. 13C) and lower right quadrants (FIG. 13D), the segments with higher HU are auto-identified (red) by software. The upper right 3D image (FIG. 13B) shows the non-interpolated data with CT auto-segmentation of the RFM filament. The RFM is auto identified and outlined in FIG. 13A while FIG. 13B shows what the RFM auto detection result looks like as seen on each slice with missing gaps due to resolution of CT.

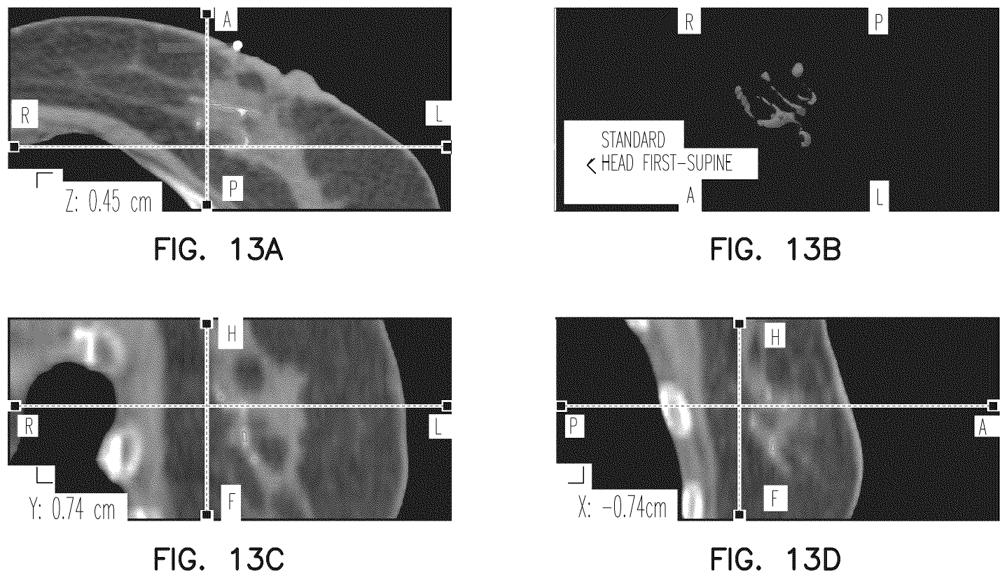

In FIGS. 14A-14D, due to CT slice resolution, the CT data for the filament has to be interpolated into a single, continuous structure so that it represents the real-world physical shape of the filament. As seen in this group of figures, the red and yellow segments form the continuous filament that outlines the tumor bed. The gaps in this series of figures are interpolated by the computer system and filled in.

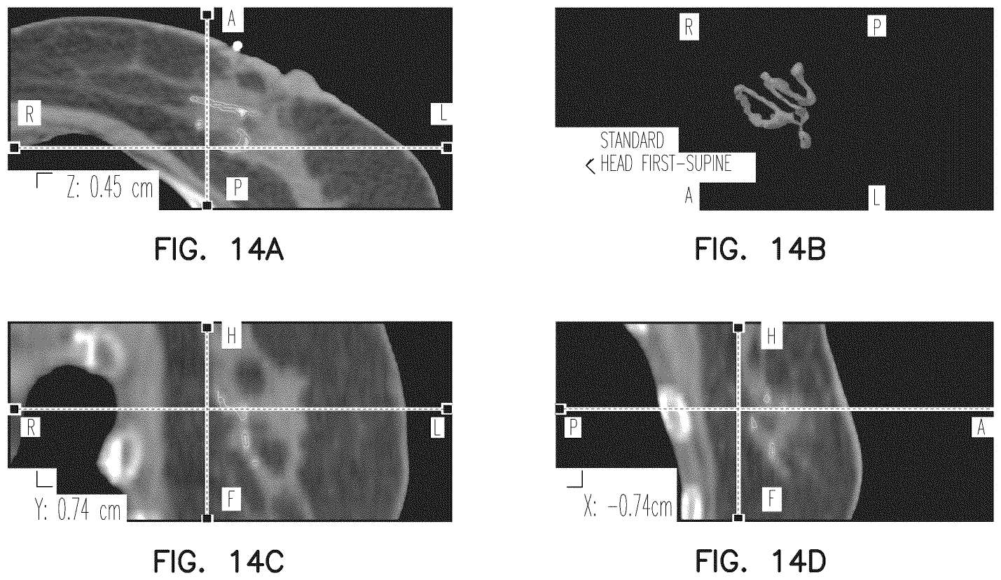

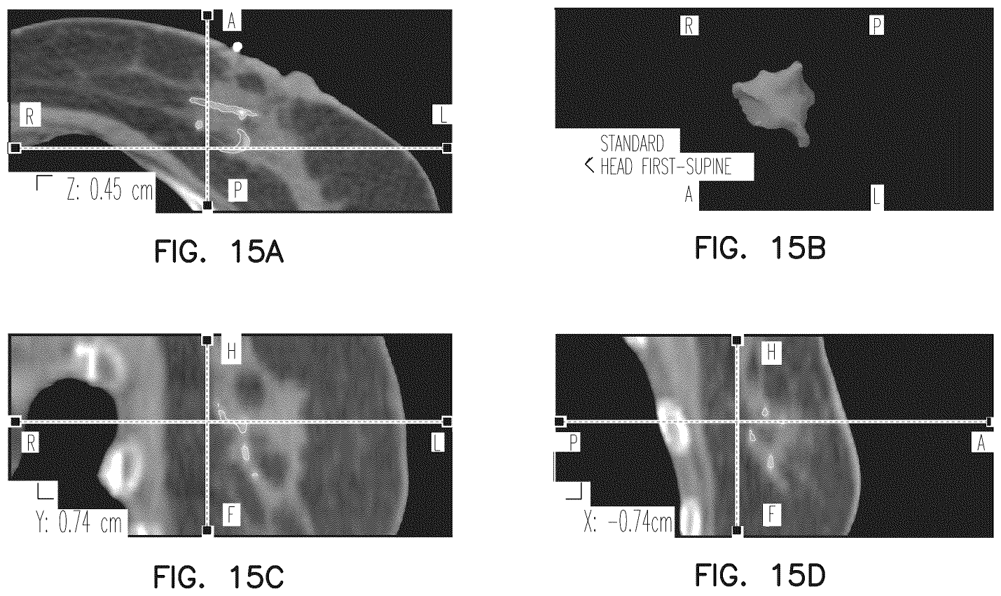

In FIGS. 15A-15D, using a contouring program, the filament is further constructed into a volumetric structure. This volumetric 3D structure is the tumor bed target for radiation.

### RFM Features

Any of the RFMs disclosed herein may have enough tensile strength to ensure that the filament may be sutured into tissue and knotted without fear of breakage. Tensile strength is used herein as the magnitude of the force required to break a filament and may be measured in Newtons or pounds. Therefore, the filament tensile strength may also be affected by the diameter of the filament. A larger diameter filament provides increased tensile strength, but if the filament is too large, then it becomes unwieldly and difficult to pull through tissue and knot. Similarly, a diameter that is too small results in a weak filament that can easily break and also that may not be radiopaque enough to visualize. Also, the RFM should be visible under x-ray or other radiographic imaging techniques. In some examples, any of the RFMs disclosed herein may be visible under low dose radiation.

The marker may be a sterile polymeric fiber that is visible using low dose radiation. The fiber can have one or more of the following properties: a fiber diameter of approximately 100 microns to approximately 1000 microns, a tensile strength greater than approximately 10 Newtons (N); a knot strength greater than approximately 5N; and/or an elongation at rupture of less than approximately 70%.

In addition to the examples disclosed above. The RFM filament can have various diameters. The diameters can range, for example, between 0.1 mm and 1 mm. Larger filaments can be developed and have diameters larger than 0.4 mm, or 1 mm or 10 mm with round or other cross-sections such as oval, square, rectangular, elliptical, etc. The tensile strength of the filament can have a range between 5 and 20 Newtons. The elongation can be greater than 50% with weight % of the radiopaque material between 5-80%. In addition, the elongation of the filament can be greater than 50%. The RFM filament can be made from various materials including a polymer. The filament may remain permanently in the body, or it may be fabricated from a bioresorbable material.

The RFM may be a filament that includes a dye or colorant such as copper phthalocyanine blue to facilitate visualization of the RFM during use where blood, tissue, or other items may render visualization of the filament difficult.

The RFM may be radiopaque because the filament is doped with a radiopaque dopant. Any dopant disclosed herein may be used. As an example barium sulfate may be the dopant in a polymer filament such as polypropylene. The barium sulfate content may be greater than 20% w/w.

An example of a RFM observable under low dose computer tomography includes a RFM observable in CT scans performed at 100 kV and 45 mAs. Using either a kV or mAs range, a low dose CT can be performed at <=120 kV and/or <100 mAs.

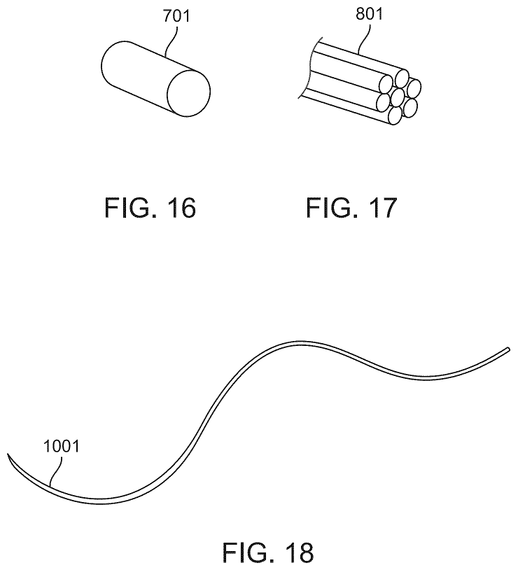

A RFM tissue marker, as disclosed herein, can be a monofilament 701 as illustrated in FIG. 16, or it may be a multifilament 801 as illustrated in FIG. 17, so that it has a low tissue reaction potential and can permanently be implanted in a patient. The filament may have a circular cross-section as illustrated in FIG. 16. Other cross-sections may be used such as oval, elliptical, square, rectangular, triangular, etc. The RFM filament can also be absorbable. As mentioned earlier the marker can be painted or sprayed on to the tissue. The RFM can also have adhesion properties. The properties of a single RFM filament may be duplicated in a multi-filament RFM as seen in FIG. 17. All of the filaments in FIG. 17 may have the same properties, or properties may be varied amongst the filaments.

The RFM may be a filament which can be attached to soft tissue with use of a needle 1001 as illustrated in FIG. 18, and standard surgeon's knots to quickly and inexpensively mark the tissue so that the integrity and location of the marked tissue can be evaluated. The marker may be placed into bone with or without a bone anchor. A needle may be coupled to one end or a needle may be included on each end of the filament.

The RFM filament may have a first portion and a second portion that are coupled to first and second portions of tissue, respectively. The first portion of the RFM filament may be configured to move independently of the second portion of the RFM filament. The first portion of the RFM filament may be configured to move with the first portion of the tissue when it is moving, and the second portion of the RFM filament may be remain stationary when the second portion of the tissue remains stationary.

In addition to marking a tumor cavity or any tissue of interest, a tissue specimen such as a tumor removed from the cavity may be marked with any of the examples of RFM disclosed herein. The RFM may be used to mark any of the six surgical planes including anterior, posterior, superior, inferior, lateral, and medial planes relative to its original orientation in the patient's body for later reference if needed.

In addition to marking a tumor cavity, a tissue specimen such as a tumor removed from the cavity may be marked with any of the examples of RFM disclosed herein. The RFM may be used to mark any of the surgical planes, such the anterior, posterior, inferior, posterior, lateral and medial planes relative to its original orientation in the patient's body for later reference if needed.

In any example, the RFM filament may be resorbable, or it may be permanent and unresorbable and it can be either threaded or attached to the tissue with mechanical means.

### RFM Implantation

As previously discussed, after certain surgical procedures, patients may require radiation therapy to irradiate any remaining cancer cells or excessively dividing cells near the site of surgery. This radiation therapy occurs after abnormal tissue is removed and the surgical cavity is closed. However, after the cavity is closed, it is extremely difficult for a radiation oncologist to gauge the actual extent of the original tumor and the subsequent tumor bed especially when there is a complex closure of the incision. This manipulation of tissue is typically referred to as tissue re-arrangement where a portion of the closure moves in various planes in a non-symmetric closure. This is particularly common in soft tissue surgical procedures. Since radiation target/treatment planning is typically performed using x-ray based imaging after the soft tissue has been closed, the imaging often does not delineate the precise location where malignant or otherwise diseased tissue was removed and when radiation therapy to the tumor bed may be required. If radiation treatment is not necessary, it still may be beneficial to mark the surgical cavity for future monitoring and follow up of the patient. Therefore, a RFM may be used to help delineate where the removed abnormal tissue was to aid the physician in directing the radiation therapy post-surgery.

An example of a marker that may be used in any of the examples above includes an elongated, flexible, continuous RFM adaptable tissue marker as illustrated in FIGS. 10 and 18. The marker can be formed into a filament with or without an attached needle that can be threaded through or otherwise attached to tissue. Since the RFM is continuous and flexible it can be deployed, attached, or threaded into a symmetric or non-symmetric shape area and/or volume. This area or volume can then be easily correlated to the radiotherapy treatment plan that closely matches the area and/or volume shape.

The filament can be self-knotted to itself and secured/anchored to tissue or it can have features that prevent itself from slipping out of position such as barbs or an anti-slip coating.

In general terms the continuous RFM filament may extend within one or more adjacent planes and/or between adjacent planes. The RFM filament can start on any of the planes by any attachment method such as by suturing, clipping, or other means. The RFM filament may extend parallel to the starting plane until an adjacent plane is reached. Then when the adjacent plane is reached, the filament may turn in a different direction along the adjacent plane, being attached again in a parallel fashion to that plane. This can continue on some or every adjacent plane as the filament reaches it and turns.

Referring back to FIG. 10, an example of a marker 302 is shown in a surgical cavity. The RFM 302 is threaded from the bottom (posterior) of the cavity 304 up to the top (anterior) of the cavity 304 in one or multiple continuous paths across a plurality of surgical planes that are stacked on top of one another and substantially parallel to one another or in some cases the planes may be transverse to one another. The RFM is threaded continuously and follows the contours of the cavity, extending continuously partially or completely around the perimeter of the cavity, optionally forming a spiral or helix around the cavity. All the planes are defined as the filament completely surrounds the cavity. The sections of the RFM filament are shown on the inside of the cavity with gaps in between, however in those gaps, the filament is still continuous, but since it is sutured into the tissue, the filament is not observable by the naked eye but will show up on a radiograph as a continuous filament.

FIG. 10 shows the filament extending from the bottom of the cavity (posterior) to the top of the cavity (anterior), but this may also be reversed from top (anterior) to bottom (posterior). It may be desirable to make sure that all the sides of the cavity are threaded from the deepest point to the highest. What may also be desirable is to only mark the portion of the cavity where the tumor was located. So, if the tumor was only at the lower portion of the surgical cavity, only that portion of the cavity would be marked and thus the upper portion or anterior portion would remain unmarked. Thus, the RFM may form a continuous band in a spiral or helix in a plurality of planes in the posterior portion of the cavity and the anterior portion of the cavity may remain marker-free. In this example one can see the filament is a running thread, weaving in and out of tissue where segments of the filament are disposed on the inner surface of the cavity and other segments are unexposed and disposed in the tissue walls surrounding the cavity. It may be desirable that one surrounds the bottom portion of the tumor bed first. A small portion of the filament tail end may be left out of the tissue and while the remainder of the filament proceeds around the posterior-most (or anterior-most) plane. Once the loop defining the posterior plane is completed and the filament tail is reached, the filament and the filament tail may be knotted together. Alternatively, a knot may be formed in the suture filament end that anchors that end in tissue without requiring knotting with another portion of the filament and prevents movement of the filament. The RFM filament may then proceed upward (or downward if you started at the anterior plane) with a running filament in a spiral pattern. The tied-off loop or knot will secure the filament at the starting end of the cavity and it will not move as it is threaded upward. A single knot or multiple knots can be self-knotted at any location around the marker depending on the tissue type to eliminate slippage of the knot and filament so the marker is firmly anchored in place. Once the filament reaches the desired height or position, the filament is tied off with another knot or knots to secure it at the opposite end, in this example the top of the cavity assuming the filament started at the bottom of the cavity. The posterior knot 306 and anterior knot 306 are shown in FIG. 10. The same result may be obtained by suturing in shorter segments instead of a continuous spiral where there are breaks or even multiple discrete rings may be used. However, this still results in having a continuous flexible filament disposed in a plurality of adjacent planes. It is also feasible that instead of having knots to tie off the loops, the RFM filament can be attached via clips or other techniques that rely on increasing friction between the filament and the tissue to reduce movement such as those disclosed herein, or a combination of clips and knots may be used. It is assumed that instead of bottom to top, the user may choose to create a path from side to side, (example lateral to medial) or in any other preferred orientation or direction.

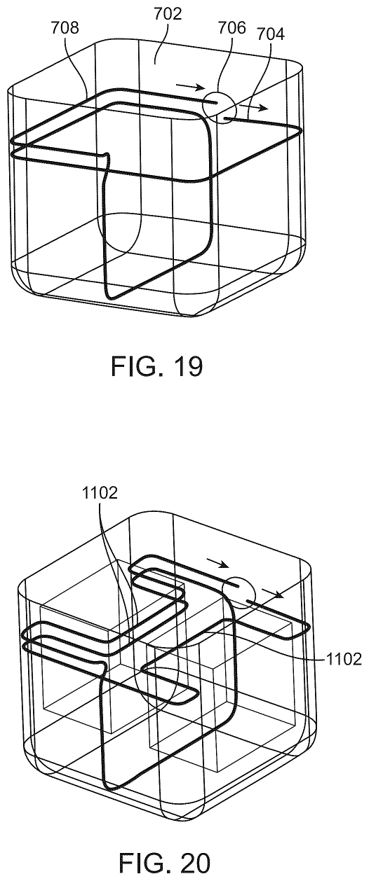

FIG. 19 shows another example of a RFM implanted in a patient's body. FIG. 19 is a perspective diagram showing the RFM filament pattern and uses cube-like openings so that the six surgical planes are easily discernable. One of skill in the art will appreciate that surgical cavities are not limited to square/rectangular cavities and any cavity shape is possible, including and not limited to oval, round and asymmetric shapes, deformed volumes and non-geometric shapes. Also, the starting points are suggested as examples. The starting point in all diagrams and ending point can be on any of the planes that are convenient for the surgeon. The following figures are merely examples.

In FIG. 19, the cavity 702 is contoured with a RFM 708 which may be any of those disclosed herein, and includes starting 704 on one of the vertical planes (leaving a filament tail to eventually tie off to) and running a continuous or discontinuous loop threading through all four adjacent planes as well as a plane in the bottom of the cavity. First, the marker starts 704 in a wall of the cavity in a vertical plane and extends a short linear distance horizontally until it passes into the adjacent vertical plane and extends along the second vertical plane in a horizontal linear continuous path. The RFM filament (also referred to herein as the filament, RFM or marker) then passes into a third wall or third vertical plane and extends along that plane in a horizontal manner until it passes into the fourth vertical plane, again in a continuous or discontinuous path then back horizontally into the original first vertical plane. The filament may extend partially along the first vertical plane up to but not past the starting point 704 where it may be sutured 706 to the tail at the start 704, or it may extend anywhere before, after or to the starting point 704 forming a loop. Also, the filament at the start 704 may be knotted to provide an anchor either alone or in conjunction with being tied to the tail. The loop may be in a plane that is parallel or transverse to the bottom of the cavity. Once the marker extends at least partially along the first vertical plane in a horizontal manner, the marker then turns downward to extend down the first vertical plane in a linear path until it hits the bottom of the cavity in the bottom plane or adjacent to it. Again this may be a continuous or discontinuous path. When the marker hits the bottom plane or is adjacent the bottom plane, the marker then turns directions again and passes along the bottom of the cavity in the bottom plane in a continuous or discontinuous manner and in a linear path to cross the bottom of the cavity until the marker comes to the third vertical plane where the marker then turns and extends upwardly in that plane and it may or may not cross over the first horizontal segment in that plane. The marker then turns and extends horizontally along the third plane, crosses into the fourth vertical plane and extends across the fourth vertical plane in a continuous or discontinuous linear path and then turns again into the first vertical plane where the marker extends in a linear continuous or discontinuous path horizontally toward the start 704 and the tail and marker may be tied together. Thus, the marker extends through the perimeters of the cavity allowing a physician to visualize the cavity under x-ray or other imaging modalities later on.

As previously mentioned, the entire path can be done in multiple segments with multiple knots joining the segments instead of just a single continuous segment and single knot. The filament can be threaded/sutured into and out of the tissue, or attached via mechanical means like clips.

Because the marker does not fill the cavity up, there is adequate space for a brachytherapy probe or radioactive elements to be disposed in the cavity as a source of radiation for post tumor removal therapy. The cavity can then be sutured closed with various walls of the cavity re-approximated with adjacent or opposed walls.

FIG. 19 depicts how the marker is originally attached to the walls of a cavity. FIG. 20 shows how the marker deforms with the tissue walls when they are apposed or otherwise re-approximated with one another.

In FIG. 20, markers 1102 in opposite vertical planes are pulled in toward one another as the tissue in those walls are apposed with one another to close the cavity. The marker position of the other filaments remain substantially the same as originally described in FIG. 19 since there is substantially no other tissue movement. The filament now outlines the tumor bed once the tissue planes have been moved.

FIG. 20 may be idealized but does emphasize that the center portion of opposing walls move inward toward one another while the outer portions of opposing walls may move inward less and thus there is corresponding movement of the marker. The marker moves with the tissue as it moves relative to other tissue where the other tissue moves or is stationary and therefore the marker in the other tissue also either moves or is stationary.

As demonstrated, the filament represents the delineated margins regardless of surgical procedure performed after tumor excision and marking of the tumor bed with the filament.

FIGS. 19 and 20 represent the same marked tumor bed. FIG. 19 is the radiographic depiction with no tissue re-approximation after tumor excision and filament placement. FIG. 20 is a radiographic example of tissue re-approximation or oncoplastic surgery after tumor excision and filament placement. As mentioned earlier this only shows two walls being moved, however this can be done with all walls.

The previous examples described above have assumed that markers are placed between the anterior and posterior planes as seen in all the patterns shown, however this is not intended to be limiting. Therefore, one of skill in the art will appreciate that the marker(s) may start, end or otherwise be placed in any plane and hence the examples are not limited to specific planes.

### NOTES AND EXAMPLES

The following, non-limiting examples, detail certain aspects of the present subject matter to solve the challenges and provide the benefits discussed herein, among others.

Example 1 is a radiopaque fiducial marker (RFM), comprising: an implantable marker element having a density ranging from about 0.8 g/cm3 to about 1.5 g/cm3 such that the implantable marker element is radiopaque and minimizes or avoids artifacts under radiologic imaging.

Example 2 is the RFM of Example 1, wherein the implantable marker element comprises an elongate filament with a dopant disposed therein.

Example 3 is the RFM of any of Examples 1-2, wherein the elongate filament or the dopant is paramagnetic or diamagnetic with an absolute value of magnetic susceptibility less than 154.

Example 4 is the RFM of any of Examples 1-3, wherein the implantable marker element has a radiodensity of between +100 to +2800 Hounsfield Units so that the implantable marker element is distinguishable from adjacent soft tissue and adjacent bony anatomy under computerized tomography.

Example 5 is the RFM of any of Examples 1-4, wherein the implantable marker element has a radiodensity of at least 100 Hounsfield Units above a radiodensity of adjacent soft tissue, or a radiodensity of at least 100 Hounsfield Units below adjacent bony anatomy so that the implantable marker element is distinguishable from the adjacent soft tissue or the adjacent bony anatomy under computerized tomography.

Example 6 is a method for marking tissue of interest, the method comprising: coupling a radiopaque fiducial marker (RFM) to the tissue of interest, wherein the RFM has a density ranging from about 0.8 g/cm3 to about 1.5 g/cm3 so that the RFM is radiopaque; and visualizing the tissue of interest demarcated by the RFM with minimal or no artifacts caused by the RFM while imaging the target tissue under radiologic imaging.

Example 7 is the method of Example 6, wherein the RFM comprises an elongate filament with a dopant disposed therein.

Example 8 is the method of any of Examples 6-7, wherein the elongate filament or the dopant is paramagnetic or diamagnetic with an absolute value of magnetic susceptibility less than 154.

Example 9 is the method of any of Examples 6-8, wherein the RFM has a radiodensity of between +100 to +2800 Hounsfield Units so that the RFM is distinguishable from adjacent soft tissue and adjacent bony anatomy under computerized tomography.

Example 10 is the method of any of Examples 6-9, further comprising: imaging the tissue of interest marked with the RFM, the RFM comprising an elongate filament that continuously extends adjacent a perimeter of the tissue of interest through one or more planes.

Example 11 is the method of any of Examples 6-10, wherein the RFM has a radiodensity of at least 100 Hounsfield Units above a radiodensity of adjacent soft tissue or a radiodensity of at least 100 Hounsfield Units below adjacent bony anatomy.

Example 12 is a radiopaque fiducial marker (RFM) comprising: a RFM filament configured to pass continuously adjacent a tissue of interest, wherein the RFM has a radiodensity of between +100 to +2800 Hounsfield Units so that the RFM is distinguishable from adjacent soft tissue and adjacent bony tissue under computerized tomography.

Example 13 is the RFM of Example 12, wherein the RFM filament is configured to pass through one or more planes to surround the tissue of interest.

Example 14 is the RFM of any of Examples 12-13, wherein the RFM has a density ranging from about 0.8 g/cm3 to about 1.5 g/cm3 such that the RFM is radiopaque but minimizes or avoids artifacts under radiologic imaging.

Example 15 is the RFM of any of Examples 12-14, wherein the RFM filament comprises an elongate RFM filament and a dopant disposed therein, and wherein the elongate RFM filament or the dopant is paramagnetic or diamagnetic with an absolute value of magnetic susceptibility less than 154.

Example 16 is a radiopaque fiducial marker (RFM), comprising: a RFM filament configured to pass continuously adjacent a tissue of interest, wherein the RFM has a radiodensity measured in Hounsfield Units, wherein adjacent soft tissue and adjacent bony anatomy has a radiodensity measured in Hounsfield Units, and wherein the radiodensity of the RFM is at least 100 Hounsfield Units above the adjacent soft tissue or the radiodensity of the RFM is at least 100 Hounsfield Units below than adjacent bony anatomy so that the RFM is distinguishable from the adjacent tissue or the adjacent bony anatomy under computerized tomography.

Example 17 is the RFM of Example 16, wherein the RFM filament is configured to pass through one or more planes to surround the tissue of interest.

Example 18 is the RFM of any of Examples 16-17, wherein the RFM has a density ranging from about 0.8 g/cm3 to about 1.5 g/cm3 such that the RFM is radiopaque but minimizes or avoids artifacts under radiologic imaging.

Example 19 is the RFM of any of Examples 16-18, wherein the RFM filament comprises an elongate RFM filament and a dopant disposed therein, and wherein the elongate RFM filament or the dopant is paramagnetic or diamagnetic with an absolute value of magnetic susceptibility less than 154.

Example 20 is a method for treating a tissue of interest, the method comprising: imaging the tissue of interest marked with a RFM filament that continuously extends adjacent a perimeter of the tissue of interest, wherein the RFM filament has a radiodensity of between +100 to +2800 Hounsfield Units.

Example 21 is the method of Example 20, wherein the RFM filament is disposed in one or more planes.

Example 22 is the method of any of Examples 20-21, wherein the RFM filament has a density ranging from about 0.8 g/cm3 to about 1.5 g/cm3 so that the marker element is radiopaque, the method further comprising visualizing the tissue of interest and adjacent tissue or adjacent bony anatomy with minimal or no artifacts caused by the RFM filament when using radiologic imaging.

Example 23 is the method of any of Examples 20-22, wherein the RFM filament comprises an elongate RFM filament and a dopant disposed therein, and wherein the elongate RFM filament or the dopant is paramagnetic or diamagnetic with an absolute value of magnetic susceptibility less than 154.

Example 24 is the method of any of Examples 20-23, wherein the RFM filament has a radiodensity of at least 100 Hounsfield Units above a radiodensity of adjacent soft tissue or the RFM filament has a radiodensity of at least 100 Hounsfield Units below adjacent bony anatomy so that the RFM filament is distinguishable from the adjacent tissue or the adjacent bony anatomy under computerized tomography.

Example 25 is a method for treating a tissue of interest, the method comprising: imaging the tissue of interest marked with a radiopaque fiducial marker (RFM) filament that continuously extends adjacent a perimeter of the tissue of interest, wherein the RFM filament has a radiodensity measured in Hounsfield Units, wherein soft tissue adjacent the tissue of interest and bony anatomy adjacent the tissue of interest has a radiodensity measured in Hounsfield Units, and wherein the radiodensity of the RFM is at least 100 Hounsfield Units above the radiodensity of the adjacent soft tissue and the radiodensity of the RFM is at least 100 Hounsfield Units below the radiodensity of the adjacent bony anatomy so that RFM is distinguishable from the adjacent soft tissue and the adjacent bony anatomy under computerized tomography.

Example 26 is the method of Example 25, wherein the RFM filament extends through one or more planes.

Example 27 is the method of any of Examples 25-26, wherein the RFM filament has a density ranging from about 0.8 g/cm3 to about 1.5 g/cm3 so that the marker element is radiopaque, the method further comprising visualizing the tissue of interest and the adjacent soft tissue or the adjacent bony anatomy with minimal or no artifacts caused by the RFM filament when using radiologic imaging.

Example 28 is the method of any of Examples 25-27, wherein the RFM filament comprises an elongate RFM filament and a dopant disposed therein, and wherein the RFM filament or the dopant is paramagnetic or diamagnetic with an absolute value of magnetic susceptibility less than 154.

Example 29 is the method of any of Examples 25-28, wherein the RFM filament has a radiodensity of between +100 to +2800 Hounsfield Units.

In Example 30, the apparatuses or methods of any one or any combination of Examples 1-29 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A radiopaque fiducial marker (RFM), comprising:
an implantable marker element comprising a radiopaque dopant containing a bismuth compound, wherein the RFM is radiopaque and minimizes or avoids artifacts under radiographic imaging.

2. The RFM of claim 1, wherein the bismuth compound comprises bismuth oxychloride (BiOCl), bismuth oxide (Bi₂O₃), or bismuth subcarbonate (Bi₂O₂(CO₃)).

3. The RFM of claim 1, wherein the implantable marker element comprises an elongate filament with the radiopaque dopant disposed therein.

4. The RFM of claim 3, wherein the elongate filament is a monofilament.

5. The RFM of claim 3, wherein the elongate filament comprises polypropylene, polyester, polyvinylidene fluoride, catgut, polyglactin, silk, or polyamide.

6. The RFM of claim 1, wherein the RFM has a density of <1.5 g/cm³.

7. The RFM of claim 1, wherein the RFM has a diameter of 100 microns to 1000 microns.

8. The RFM of claim 1, wherein the RFM is configured to remain permanently in tissue of a patient's body.

9. The RFM of claim 1, further comprising a dye or colorant configured to facilitate visualization of the RFM when disposed in tissue of a patient's body.

10. The RFM of claim 1, further comprising a needle coupled to the RFM, the needle configured to facilitate attachment of the RFM to tissue in a patient's body.

11. A radiopaque fiducial marker (RFM), comprising:
an implantable marker element comprising a radiopaque dopant containing a compound containing an atom having an atomic number of 83, wherein the RFM is radiopaque and minimizes or avoids artifacts under radiographic imaging.

12. The RFM of claim 11, wherein the radiopaque dopant comprises bismuth oxychloride (BiOCl), bismuth oxide (Bi₂O₃), or bismuth subcarbonate (Bi₂O₂(CO₃)).

13. The RFM of claim 11, wherein the implantable marker element comprises an elongate filament with the radiopaque dopant disposed therein.

14. The RFM of claim 13, wherein the elongate filament is a monofilament.

15. The RFM of claim 13, wherein the elongate filament comprises polypropylene, polyester, polyvinylidene fluoride, catgut, polyglactin, silk, or polyamide.

16. The RFM of claim 11, wherein the RFM has a density of <1.5 g/cm³.

17. The RFM of claim 11, wherein the RFM has a diameter of 100 microns to 1000 microns.

18. The RFM of claim 11, wherein the RFM is configured to remain permanently in tissue of a patient's body.

19. The RFM of claim 11, further comprises a dye or colorant configured to facilitate visualization of the RFM when disposed in tissue of a patient's body.

20. The RFM of claim 11. further comprising a needle coupled to the RFM, the needle configured to facilitate attachment of the RFM to tissue in a patient's body.
